(19) United States
(12) Patent Application Publication
(10) Pub. No.: US 2011/0150934 A1
(43) Pub. Date: Jun. 23, 2011

(54) Ovarian Tumor Tissue Cell Preparations/Vaccines for the Treatment/Inhibition of Ovarian Tumors and Ovarian Cancer

(75) Inventor: Mark A. Suckow, Granger, IN (US)

(73) Assignee: University of Notre Dame, Notre Dame, IN (US)

(21) Appl. No.: 12/641,633

(22) Filed: Dec. 18, 2009

(51) Int. Cl. A61K 39/00; C12N 5/00; C12N 5/071; A61K 35/12; A61P 35/00; A61P 37/00

(52) U.S. Cl. 424/277.1; 435/325; 435/366; 424/573

(57) ABSTRACT

Disclosed are compositions comprising non-viable ovarian tumor cells and/or ovarian cancer tissue cells useful as a vaccine for human ovarian cancer and/or for the inhibition of human ovarian tumor growth. Methods for preparing the ovarian cancer and/or tumor vaccines that comprise these compositions are also provided, as well as methods of using the vaccines in the treatment and/or inhibition of tumor growth, and particularly ovarian tumor growth and ovarian cancers. The preparations may be defined as vaccines comprising human ovarian tumor tissue cells and/or human ovarian cancer cells. Preparations comprising the ovarian tissue cells together with an adjuvant are also provided. The ovarian tumor and/or ovarian cancer vaccines are demonstrated to significantly reduce the incidence of autochthonous ovarian cancer.

### STATEMENT OF JOINT RESEARCH AGREEMENT

In compliance with 37 C.F.R. §1.71(g)(1), disclosure is herein made that the claimed invention was made pursuant to a Joint Research Agreement as defined in 35 U.S.C. §103© (3), that was in effect on or before the date the claimed invention was made, as a result of activities undertaken within the scope of the Joint Research Agreement, by or on the behalf of the University of Notre Dame and Cook Biotech, Inc. (West Lafayette, IN.).

### BACKGROUND

1. Field of the Invention

The present invention relates to the field of ovarian tumor inhibition preparations and ovarian cancer treatment preparations and methods of treating, inhibiting and immunizing an animal for ovarian cancer. The invention also relates generally to the field of ovarian tumor and cancer vaccines, and more particularly to anti-ovarian tumor and anti-ovarian cancer vaccines. The invention also relates to the field of methods for preparing compositions useful for treating ovarian cancer and inhibiting ovarian tumor growth, as methods for preparing compositions that include non-viable preparations of ovarian cancer and/or ovarian tumor tissue cells are provided.

2. Background

Human ovarian cancer is a common gynecological malignancy. Ovarian cancers shed cells into the naturally occurring fluid within the abdominal cavity. These cells can implant on other abdominal (peritoneal) structures, including the uterus, urinary bladder, bowel and the lining of the bowel wall (omentum).

Stage I ovarian cancer is confined to one or both ovaries. In Stage II, the ovarian cancer has spread to pelvic but not the abdominal organs. In Stage III, the ovarian cancer has spread to abdominal organs and in Stage IV, the cancer has spread to distant sites, for example the lung, brain, or lymph nodes in the neck. Within these stages there are subcategories that are identified based on tumor size, node involvement and metastatic status. Thus a tumor can be a IIA, which describes a tumor that has spread and attached to the uterus, where as a IIB tumor describes a tumor that has in addition spread to other pelvic tissues, but with no cancer cells in the ascites or peritoneum, and so on. In addition to tumor staging, ovarian tumors, a type of epithelial tumor, can also be graded. Grade refers to the character of the cells of the tumor. Grade 1 is the least malignant with well-differentiated cells, Grade 2 is intermediate with moderately differentiated cells, and Grade 3 is the most malignant with poorly differentiated cells.

Ovarian cancer generally has a poor prognosis. It is estimated that one woman in 100 will die from this cancer in the United States. Ovarian cancer is disproportionately deadly because it lacks any clear early detection or screening test, meaning that most cases are not diagnosed until they have reached advanced stages. For example, more than 60% of patients presenting with this cancer already have Stage III or Stage IV ovarian cancer. The five-year survival rate for all stages of ovarian cancer is only 45.5%. Because of its aggressive nature, new approaches to the prevention and/or treatment of ovarian cancer are needed.

### SUMMARY

The present invention, in a general and overall sense, relates to ovarian cancer tissue and/or ovarian tumor tissue cell preparations.

In some embodiments, a pharmaceutical preparation useful in the inhibition and/or treatment of ovarian cancer and ovarian tumors is provided. In some embodiments, the pharmaceutical preparations comprise a non-viable preparation of ovarian cancer tissue and/or ovarian tumor tissue cells. In some embodiments, the ovarian cancer tissue and/or ovarian tumor tissue cells are human ovarian cancer tissue cells and/or human ovarian tumor tissue cells. These pharmaceutical preparations may also comprise a pharmaceutically acceptable carrier, such as saline. However, any variety of pharmaceutically acceptable carriers may be used as recognized by those of ordinary skill in the pharmaceutical arts.

In other embodiments, the invention comprises methods for treating and/or inhibiting ovarian tumor growth and for inhibiting or treating ovarian cancer, particularly human ovarian tumor growth and human ovarian cancer. In some embodiments, the , method for treating and/or inhibiting ovarian tumor growth comprises administering an effective dose of a non-viable preparation of ovarian tumor cells to a patient in need thereof, and inhibiting ovarian tumor growth in the patient. The method may include, for example, multiple additional steps and subsequent treatments with the non-viable ovarian tumor preparations, as may be needed or desired in the treatment of the individual patient. It is envisioned, for example, that at least one booster shot of the non-viable ovarian tumor cells will be required as part of a treatment method as described herein. In addition, it may be beneficial/advantageous to the patient's treatment to provide a combination therapeutic approach to the treatment of the patient having ovarian cancer and/or an ovarian tumor. Thus, it is envisioned that a multiple modality of medicaments may be administered to the patient together with, followed by, or before administration of the non-viable ovarian tumor tissue cell preparations and/or non-ovarian cancer tumor tissue cell preparations described herein.

Some embodiments of the invention may be described as comprising a composition comprising a non-viable preparation of whole ovarian tumor or ovarian cancer tissue cells. For example, the preparation may include stromal (connective and other tissue) components with the ovarian tumor tissue, the whole ovarian tumor tissue cell preparation of non-viable cells being capable of inhibiting ovarian tumor cell proliferation and capable of inhibiting growth of an ovarian tumor.

In another embodiment, the non-viable whole ovarian tumor and/or cancer tissue cell preparation comprises a glutaraldehyde processed ovarian tumor and/or ovarian cancer tissue cell preparation. In some embodiments, the non-viable whole ovarian tumor cell and/or cancer tissue cell preparation is rendered non-viable through a chemical treatment, such as by treatment with potassium thiocyanate, glutaraldyhyde, or other chemical agent.

As used in the description of the present invention, the ovarian tumor tissue of the compositions as part of a vaccine or other immuno-protective preparation is described as non-viable, that is, a preparation comprised of ovarian tumor tissue that is unable to replicate and/or give rise to other ovarian tumor cells or tissue under normal, physiologically supportive conditions. For example, normal, physiologically supportive conditions may be described as under ordinary mammalian cell culture conditions or when placed and/or injected into an animal/human. An ovarian tumor tissue may be rendered non-viable according to many techniques, including radiation treatment, chemical lysis, ultrasound lysis, freezing, freeze-drying, chemical processing (glutaraldehdye, etc), exposure to particular heating temperatures, or other techniques known to those of skill in the art. Thus, the preparations and compositions that include the ovarian tumor adjuvant when administered together with the ovarian tumor antigen and/or antigens would not be expected to be capable of giving rise to or creating an ovarian tumor and/or ovarian tumor growth in the animal.

As used in the description of the present invention, an ovarian cancer and/or tumor vaccine relates to a preparation and/or composition that comprises a variant or derivative of an antigen or antigens of an ovarian tumor and/or ovarian cancer tissue, will result in an immunologically detectable protective or immune response against ovarian tumor growth and/or ovarian cancer in an animal, such as in a human patient. Thus, and according to some embodiments of the invention, the ovarian cancer preparations and/or vaccines disclosed herein provide variants or derivatives of ovarian cancer cells and tissue that are presented to the body in order to induce an immune response against ovarian cancer cells. The induced response allows the immune system to attack, kill and remove ovarian cancer cells resulting in the prevention and/or treatment of ovarian cancer and/or ovarian tumor growth.

In another embodiment, the composition and/or preparation, particularly a pharmaceutically acceptable preparation for use in the inhibition and/or treatment of ovarian cancer and/or an ovarian tumor cell growth, is provided as a vaccine suitable for administration to an animal, and in particular, suitable for administration to a human patient having or at risk of developing an ovarian tumor or ovarian cancer.

The present preparations of non-viable ovarian tumor tissue, non-viable ovarian tumor cells and/or ovarian cancer tissue or cell preparations from this tissue may be combined with any other number of adjuvants and/or carrier solutions. For example, the cell and/or tissue preparations may be combined with any physiologically compatible solution, such as saline, or other physiologically compatible solution.

In another embodiment, the tumor tissue comprises a non-viable preparation of non-human ovarian tumor tissue. In another embodiment, the stromal tissue comprises non-viable tumor stromal non-human tissue.

In some embodiments, an effective dose of the non-viable ovarian tumor tissue cell and/or ovarian cancer tissue cell preparation comprises about 5×10⁶ non-viable ovarian cancer and/or non-viable ovarian tumor cells. In some embodiments, for example, non-viable ovarian tumor tissue cells and/or non-viable ovarian cancer cells may be prepared by treating the ovarian cancer/tumor cells with a chemical agent, such as glutaraldyhyde, so as to provide a GFT vaccine of ovarian tumor and/or cancer cells that have been rendered non-viable.

In another embodiment, the method further comprises a second administration (“booster) of an effective dose of the non-viable and/or inactivated (processed) ovarian tumor and/or ovarian cancer tissue cell preparation, the preparation being capable of inhibiting ovarian tumor cell proliferation and/or inhibiting ovarian tumor growth within about 7 days after the first administration. The inhibition of ovarian tumor cell proliferation and/or inhibition of ovarian tumor growth can be observed by a reduction in the measurable size of a tumor mass on the animal. Such measurements may be made by any number of techniques known to those of skill in the medical arts, such as by caliper measurement, or simply by manual examination and/or assessment of the tumor mass. In addition, it is envisioned that growth and/or size of a tumor mass and/or inhibition of ovarian cancer and/or tumor growth may be monitored and assessed by any variety of radiological techniques. For example, an area of the animal under assessment/treatment may be examined and/or monitored by Magnetic Resonance Imaging (MRI).

In other embodiments, methods of preparing a non-viable ovarian cancer cell and/or ovarian tumor cell vaccine, as part of a pharmaceutical preparation, are provided. One embodiment includes a method of preparing a non-viable ovarian tumor and/or cancer tissue preparation capable of preventing ovarian tumor and/or ovarian cancer growth and occurrence comprising: obtaining an ovarian tumor tissue to provide ovarian tumor and/or cancer tissue cells; exposing the ovarian tumor and/or cancer tissue cells to a treatment capable of rendering said ovarian tumor tissue and/or ovarian cancer cells non-viable to provide a non-viable ovarian tumor and/or cancer preparation, wherein the non-viable ovarian tissue preparation is capable of inhibiting ovarian tumor growth. For example, the preparation may be rendered non-viable by a treatment with glutaraldehdye, or other chemical agent, for a period of time. In some embodiments, the chemical treatment comprises incubating the ovarian tumor and/or ovarian cancer tissue/cells in about 2.5% glutaraldehdye (v/v) at about 37° C. for about 120 minutes. In some embodiments the non-viable ovarian tissue preparation comprises a vaccine. In particular embodiments, the ovarian tumor tissue is human ovarian tumor tissue. In some embodiments, the method further includes the steps of combining the non-viable ovarian tumor preparation with a pharmaceutically acceptable carrier solution suitable for use in a vaccine preparation, and providing a sterile, non-viable ovarian tumor and/or ovarian cancer formulation. These formulations are in some embodiments suitable for injection.

In some embodiments of the treatment methods, the animal is a human or non-human.

Accordingly, it is envisioned that the various preparations/compositions, methods and vaccines of the present invention may be used for both veterinary and human applications.

The following abbreviations are used throughout the description of the present invention:

GFT—Glutaraldehyde Fixed Tumor;

LW Rat—Lobund-Wistar rat;

MEM—Modified Eagle's Medium;

PAIII—Prostate Adenocarcinoma III Cell Line from LW rats;

SIS—Small Intestinal Submucosa;

### BRIEF DESCRIPTION OF THE FIGURE

FIG. 1—demonstrates the inhibition of ovarian carcinoma tumors in a mouse model. The tissue vaccine is demonstrated to inhibit growth of human ovarian carcinoma cells by 90%. (Control: Hatched Bar (ES-2 cells mixed with splenocytes from mice vaccinated with saline only); GFT (ES-2 cells mixed with splenocytes from mice vaccinated with GFT vaccine): solid bar.

### DETAILED DESCRIPTION

### Definitions

The term “stroma” refers to a whole cell mixture comprising animal (human or non-human) supportive or connective tissue characteristic of that tissue located in or around a tissue or organ, particularly that connective and/or supportive tissue located in or around a tumor tissue or whole tumor as found in vivo, i.e., in the body. Stromal preparations may not be characterized by a single type or species of cells or proteins. For example, they can be instead characterized by a mixture of diverse antigenic species characteristic of a whole stromal tissue preparation as observed in vivo in association with a whole organ or tumor.

The term “tissue preparation” refers to a heterologous mixture of tumor cell and non-tumor cell tissue. The non-tumor cell tissue can comprise, for example, connective tissue, stroma, blood, serum, bone cells, vessels, or any other animal (human or non-human) tissue other than tumor cells. The tissue preparation comprises a diverse mixture of defined and undefined antigenic species, and is comprised of antigens present on the surface and inside of whole tumor and associated (connective tissue) non-tumor tissue in a disrupted or intact cell form. A tissue vaccine of embodiments disclosed herein can include whole cells, cell lysates, tissue preparations that include tumor tissue and other tissues, such as connective and supporting tissues (stroma), etc. The term is not intended to be defined as an isolated cellular component or protein, or finite number of strictly enumerated antigenic species characteristic of a tumor cell or a connective tissue alone. Hence, as used herein, the tissue preparation and vaccines prepared therefrom or method of using employing them presents numerous targets (antigenic species) that induce an immunogenic response to a multiplicity of tumor tissue and connective tissue antigenic species. A broad spectrum antigenic immune response can thus be elicited in an animal (human or non-human) vaccinated with the tissue preparations, and can provide the anti-tumor activity described herein.

The term “tumor” refers to a combination of neoplastic tissue and associated supporting stroma and connective tissue.

The term “vaccine” refers to a preparation that contains components (antigenic species) capable of stimulating an immune response in an animal (human or non-human).

The term “GFT vaccine” refers to a tissue preparation that comprises a combination of tissue and stromal antigenic species characteristic of a tumor tissue and associated connective tissue that has been processed with glutaraldyhyde and is capable of demonstrating the tumor inhibiting activity of the glutaraldehyde processed tissue preparations described herein.

The term “PTE vaccine” refers to a tissue preparation that possesses a combination of tissue and stromal antigenic species characteristic of a tumor tissue and connective tissue that has been processed with potassium thiocyanate and is capable of demonstrating the tumor cell inhibiting activity of the potassium thiocyanate processed preparations and extracts described herein.

The term “xenogeneic” refers to a tissue or other material that is obtained from a source that is distinct from another, such as not having been obtained from the same species of animal (human vs. mouse), or same type of animal tissue (heart vs. lung).

The presently described tissue preparations, compositions and methods provide anti-cancer and anti-tumor vaccines that prevent and/or inhibit ovarian cancer and tumor growth in vivo.

Because metastatic forms of cancer are a complex mixture of neoplastic cells, connective tissue cells and matrix, the present multivalent vaccines capture the greatest range of relevant antigens, and therefore are of significant clinical utility. In this regard, the tissue vaccines of embodiments disclosed herein are made of harvested tumor material, and as such, are composed of a rich antigenic menu. In addition, the tissue vaccines provided herein are well tolerated by the animal/patient in vivo.

In some embodiments, processed tissue preparations comprise a whole tumor tissue and connective (stromal) tissue sample that has been treated with glutaraldehyde-(GFT) or potassium thiocyanate (PTE).

Without being bound by theory, it is believed that xenogeneic vaccines may allow the immune system to overcome tolerance to self-antigens expressed by tumors, thus stimulating a vigorous immunity to homologous antigens. In this way, xenogeneic vaccines may have an advantage over autologous or even allogeneic vaccines. The vaccine preparations are mixtures which contain a variety of potent antigens. In the case of prevention, the immune systems of vaccinated animals can rapidly respond to preneoplastic lesions and effectively target occasional cancer cells as they develop. In the case of treatment, the immune system faces the much greater challenge of targeting an enormous number of active cells which can induce immune tolerance and quickly alter phenotype to adapt to selective pressures from treatment. The vaccines also include connective tissue components which are not neoplastic but which can be altered by cytokine or other signals from the neoplastic cells to organize needed connective tissue and stromal infrastructure for tumor support, growth and progression. Because these connective tissue components are not neoplastic, they cannot alter their immunophenotype as easily as neoplastic cells in order to evade an immune response resulting from vaccination with a vaccine directed against these components. In this way, vaccination against tumor connective tissue and stromal components allows a protective immune response that the tumor cannot escape by rapidly altering immunophenotype, an escape mechanism commonly employed by neoplastic cells.

In some embodiments, the vaccine preparation can be described as comprising a tumor tissue vaccine or a conditioned extracellular matrix (ECM) vaccine.

Any non-toxic, inert and effective carriers can be used to formulate compositions of embodiments disclosed herein. Well known carriers used to formulate other therapeutic compounds for administration to humans particularly will be useful in the compositions of embodiments disclosed herein. Pharmaceutically acceptable carriers, excipients and diluents in this regard are well known to those of skill, such as those described in the MERCK INDEX, 11th Ed., Budavari et al., Eds., Merck & Co., Inc., Rahway, N.J. (1989), which is incorporated by reference herein in its entirety. Examples of such useful pharmaceutically acceptable excipients, carriers and diluents include, without limitation, distilled water, physiological saline, Ringer's solution, dextrose solution, Hank's solution and DMSO. Sterile formulations, such as those described in, without limitation, Mantile et al., J. Biol Chem. 268: 20343-20351 (1993), which is incorporated by reference herein for its teachings regarding the same, can also be used.

In further embodiments, the compositions and methods disclosed herein can be used in conjunction with additional treatments including, without limitation, surgical intervention, radiation therapy, hormonal therapy, immunotherapy, chemotherapy or cryotherapy.

### EXAMPLES

The present example sets forth the materials and methods employed in some of the embodiments of the invention, and as used throughout the description of the present invention.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### Example 1

### Ovarian Cancer Inhibition In Vivo

The present example demonstrates the utility of the present invention for inhibiting the growth of ovarian tumors and for treating human ovarian cancer.

Studies were undertaken to evaluate the efficacy of tissue vaccines in the treatment of ovarian cancer. A human ovarian carcinoma cell line (ES-2; American Type Culture Collection, Manassas, Va.) was used for these studies. In culture, the cells were grown in Dulbecco's Modified Eagle's Media with fetal bovine serum added to 10% concentration.

To create ES-2 cell tumors, 6×10⁶ ES-2 cells were administered subcutaneously in a volume of 0.25 ml of media over the flank of immunodeficient nude mice (Hsd: Athymic Nude-Foxnl^{um}/Foxln⁺, 6-8 week old female; Harlan Laboratories, Inc., Indianapolis, Ind.). After 18 days, tumors weighing approximately 3-4 grams were harvested following euthanasia of the animals. From the harvested tumors, tissue vaccine was prepared as previously described in Suckow M A, Rose E D, Wolter W R, Sailes V, Jeffrey R, Tenniswood M., Prevention of human PC-346C ovarian cancer growth in mice by a xenogencic vaccine. Cancer Immunology Immunotherapy, 56:1275-1283 (2007), which is incorporated by reference herein for its teachings regarding the same. Briefly, the tumor tissue was finely minced using scissors to create a cell suspension in media. The cell suspension was incubated in 2.5% glutaraldehyde (v/v) at 37° C. for 2 hours and then washed thoroughly with saline to produce the tissue vaccine (GFT).

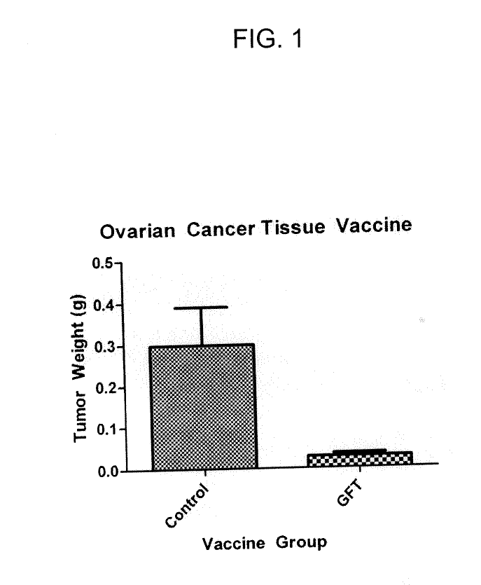

Two syngeneic immunocompetent mice (Balb/C, 6-6 week old female; Harlan Laboratories, Inc.) were vaccinated once subcutaneously in the flank with 1×10⁶ glutaraldehyde-fixed tumor cells (GFT vaccine) in a volume of 020 ml media. Two mice were similarly vaccinated with saline only as a control. A boost was administered 4 days later. Fourteen days after the first vaccination, mice were euthanized and their spleens aseptically harvested. The spleens were dissociated into medium using a screen and co-incubated for 3 hours at 37° C. with ES-2 cells (2:1 ratio of splenocytes to tumor cells). Groups of 7 nude mice were then administered subcutaneously a suspension containing 1.5×10⁶ mixed ES-2 cells and splenocytes. Mice were euthanized 21 days later and the tumors weighed. Results shown in FIG. 1 demonstrate that a significant (ρ≦0.05) reduction in mean tumor weight was noted in mice receiving ES-2 cells mixed with splenocytes from mice vaccinated with the GFT vaccine, compared to mice receiving ES-2 cells mixed with splenocytes from mice vaccinated with saline only.

While not intending to be limited to any particular mechanism of action and/or theory, this protective response can be mediated by Th1 immunity.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by embodiments disclosed herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A composition comprising non-viable ovarian tumor cells or ovarian cancer tissue cells, said composition being capable of inhibiting ovarian tumor cell proliferation.

2. The composition of claim 1 comprising glutaraldehdye processed ovarian tumor or ovarian cancer cells.

3. The composition of claim 1 comprising potassium thiocyanate processed ovarian tumor tissue or ovarian cancer cells.

4. The composition of claim 1, wherein said composition is a vaccine.

5. The composition of claim 1 wherein said ovarian tumor tissue cells comprise human ovarian tumor tissue and ovarian cancer cells.

6. The composition of claim 1 wherein said ovarian cancer tissue cells are human ovarian cancer tissue cells.

7. A method of inhibiting ovarian tumor growth comprising administering an effective amount of a composition of claim 1.

8. The method of claim 7, wherein an effective amount comprises about 5×10⁶ non-viable human ovarian tumor tissue cells.

9. The method of claim 8 wherein the method comprises administering a second booster dose of the composition of claim 1.

10. The method of claim 7, wherein the non-viable ovarian cancer cells are non-viable human ovarian cancer cells.

11. A method for immunizing an animal against ovarian cancer or ovarian tumor growth comprising:
administering an effective amount of the composition of claim 1; and
inhibiting de novo tumor growth.

12. The method of claim 11, wherein said animal is a human.

13. The method of claim 11, wherein said composition comprises non-viable human ovarian tumor tissue and human ovarian cancer cells.

14. The method of claim 11, wherein said composition comprises glutaraldehdye non-viable human ovarian tumor tissue and human ovarian cancer cells.

15. A method of preparing a non-viable ovarian tumor tissue preparation capable of preventing ovarian tumor growth and occurrence comprising:
obtaining a human ovarian tumor tissue to provide ovarian tumor tissue cells; and
exposing the ovarian tissue cells to a treatment capable of rendering said ovarian tumor tissue cells non-viable to provide a non-viable ovarian tumor preparation,
wherein the non-viable ovarian tissue preparation is capable of inhibiting ovarian tumor growth.

16. The method of claim 15, wherein said vaccine preparation comprises about 5×10⁶ non-viable human ovarian carcinoma cells of human cell line ES-2.

17. The method of claim 15, wherein the non-viable ovarian tumor preparation comprises a vaccine.

18. The method of claim 15, wherein the ovarian tumor tissue is human ovarian tumor tissue.

19. The method of claim 15 further comprising the steps of:
combining the non-viable ovarian tumor preparation with a pharmaceutically acceptable carrier solution suitable for use in a vaccine preparation; and
providing a sterile non-viable ovarian tumor formulation.

20. The method of claim 19, wherein the non-viable ovarian tumor formulation is suitable for injection.
